(19) United States
(12) Patent Application Publication
(10) Pub. No.: US 2005/0281769 A1
(43) Pub. Date: Dec. 22, 2005

(54) Cosmetic composition comprising a polymer

(76) Inventor: Béatrice Toumi, Verrieres Le Buisson (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/150,162

(22) Filed: Jun. 13, 2005

Related U.S. Application Data
(60) Provisional application No. 60/582,805, filed on Jun. 28, 2004.

(30) Foreign Application Priority Data
Jun. 11, 2004 (FR) 0406370

(51) Int. Cl. A61K 7/06; A61K 7/11

(52) U.S. Cl. 424/70.11

(57) ABSTRACT

A cosmetic skincare or makeup composition comprising a dispersion of particles of at least one grafted ethylenic polymer in at least one liquid fatty phase, wherein the at least one grafted ethylenic polymer is present in an amount sufficient to allow the composition to form a deposit comprising a complex storage modulus E* of less than or equal to 200 MPa. Also disclosed is a process for obtaining a deposit on skin comprising applying such a composition to the skin, wherein the deposit on the skin has at least one property chosen from good adhesion, comfort and good transfer resistance.

This application claims benefit of U.S. Provisional Application No. 60/582,805, filed Jun. 28, 2004, the contents of which are incorporated herein by reference. This application also claims benefit of priority under 35 U.S.C. § 119 to French Patent Application No. 04 06370, filed Jun. 11, 2004, the contents of which are also incorporated by reference.

The present disclosure relates to a cosmetic composition comprising a particular polymer and intended for application to the skin of human beings.

The composition according to the present disclosure may be a makeup composition and/or a care composition for the skin. In at least one embodiment, the composition is a makeup composition. The makeup composition disclosed herein may be a foundation, an eyeshadow, a blusher, a concealer, an eyeliner or a body makeup product. In at least one embodiment, the composition is a foundation composition.

The care composition may be a face and body skincare product, for example, a sun product, or a skin coloring product, for example, a self-tanning product.

Foundation compositions may be commonly used to give the skin, for example, the face, an aesthetic color. These makeup products generally comprise oils, pigments and/or fillers and optionally additives, such as cosmetic or dermatological actives.

These compositions, when they are applied to the skin, may exhibit the disadvantage of transferring, i.e., of undergoing at least partial deposition, leaving marks, on certain substrates with which they may be contacted, for example, a glass, cup, cigarette, item of clothing, or the skin. A consequence of this may be mediocre persistence of the applied film, making it necessary regularly to renew the application of the lipstick or foundation composition. Moreover, the appearance of these unacceptable marks, for example, on blouse collars, may put certain users off using this type of makeup.

Furthermore, the sebum excreted by the skin over the course of time may also alter the properties of the makeup. For example, sebum generally does not promote the adhesion of the makeup to the skin, and the transfer of the makeup may be even greater, giving rise to a substantial loss of the makeup remaining on the skin.

“Transfer-free” skin makeup compositions are therefore sought which exhibit the advantage of forming a transfer-resistant deposit, for example, in the presence of sebum, and compositions which do not undergo at least partial deposition onto substrates with which they are contacted, for example, glass, clothing, cigarettes, fabrics.

For the purpose of enhancing the persistence of makeup products the use of film-forming polymers is known. For example, U.S. Pat. No. 6,074,654 and PCT Publication No. WO 02/067877 disclose the use of silicone resins.

However, certain polymers for obtaining non-transfer properties are not always well suited for application to the skin. This is because the facial skin is subjected to numerous movements and it is necessary for the makeup film deposited to be able to withstand these stresses without fissuring or cracking, and not least without detaching from the skin. Not all film-forming polymers, though, allow a satisfactory deposit to be obtained on the skin, for example, when they are present in a large amount in the deposit obtained after the full drying of the composition that has been applied to the skin. The polymer may then exhibit poor adhesion to the skin, giving rise to poor hold of the makeup. Moreover, the polymer may give rise to pulling on the skin, thereby making the makeup uncomfortable for the user.

Therefore, it would be desirable to provide a cosmetic product which allows a deposit, for example, a makeup deposit, to be obtained on the skin that exhibits at least one property chosen from good adhesion, good cohesion under mechanical stress, good comfort properties, and good non-transfer properties, for example, in the presence of sebum.

The present inventors have discovered that it may be possible to obtain such a composition by using a grafted ethylenic polymer as disclosed herein.

More specifically, disclosed herein is a cosmetic makeup or skincare composition, comprising a dispersion of particles, for example solid particles, of at least one grafted ethylenic polymer in at least one liquid fatty phase as described below.

The at least one grafted ethylenic polymer is, for example, chosen such that, when it is present in sufficient amount in the composition, the composition can form a deposit having a complex storage modulus E* of less than or equal to 200 MPa, for example, ranging from 0.1 MPa to 200 MPa

Also disclosed herein is a process of making up and/or caring for the skin comprising applying to keratin materials, for example, the skin, a cosmetic composition comprising a dispersion of particles of at least one grafted ethylenic polymer in at least one liquid fatty phase, wherein the at least one grafted ethylenic polymer is present in an amount sufficient to allow the composition to form a deposit comprising a complex storage modulus E* of less than or equal to 200.

The present disclosure additionally provides for the use, in a cosmetic composition, of a dispersion of particles (for example, solid particles) of a grafted ethylenic polymer in dispersion in a liquid fatty phase, wherein the grafted ethylenic polymer is present in an amount sufficient to allow the composition to form a deposit having a complex modulus E* of less than or equal to 200 MPa, for obtaining a deposit, such as a makeup deposit, on the skin that exhibits good adhesion and/or is comfortable and/or exhibits good transfer resistance, for example, in the presence of sebum.

The complex modulus E* of the deposit obtained with the composition disclosed herein is determined according to the measuring protocol described below.

Method of Measuring the Complex Modulus E* of a Film

A Teflon plate is coated with an amount of the composition so as to obtain, after drying for 7 days on a thermostatted plate at 35° C., a deposit with a thickness ranging from 100 to 150 μm. Subsequently a sample 10 mm wide and 15 mm long is cut from the dry film.

This sample is used to carry out viscoelasticity measurement tests with a dynamic spectrometric apparatus of DMTA (Dynamic and Mechanical Temperature Analysis) type, for example the model DMA2980 from TA Instruments.

A tensile stress is imposed on the sample. The sample undergoes a static force, for example of 0.01 N, superimposed on which there is a sinusoidal displacement of, for example, ±8 μm at a frequency of 1 Hz. Operation is therefore in the linear range, under low levels of deformation. This stress is carried out on the sample at temperatures ranging from −50° C. to +100° C., with a temperature change of 3° C. per minute. For example, the sample is maintained for 5 minutes at an isotherm of −50° C.

A measurement is then made of the complex modulus E*=E′+iE″ of the film under test, as a function of temperature.

From these measurements, the dynamic storage modulus E′ (also called Young's modulus) and dynamic loss modulus E″ are deduced, and also the damping power: tgδ=E″/E′.

The complex modulus E* of the deposit (film) is the norm of the complex number E*=\sqrt{(E′²+E″²)}.

The composition according to the present disclosure may be, for example, capable of forming a deposit, for example, a film, having a complex storage modulus E* of less than or equal to 150 MPa, for example, ranging from 0.1 MPa to 150 Mpa; or less than or equal to 100 MPa, for example, ranging from 0.1 MPa to 100 MP; or less than or equal to 50 MPa, for example, ranging from 1 MPa to 50 MPa.

The composition according to the present disclosure may be, for example, capable of forming a deposit, for example, a film, having a damping power tgδ of greater than or equal to 0.7, for example, ranging from 0.7 to 2; such as greater than or equal to 0.9, for example, ranging from 0.9 to 2; or such as greater than or equal to 1.1, for example, ranging from 1.1 to 2. The damping power of the deposit is determined in accordance with the protocol described above.

In at least one embodiment, the composition according to the present disclosure may be, for example, capable of forming a deposit having at least one glass transition temperature of ranging from 0° C. to 40° C.

In at least one embodiment, the transition temperature of the deposit (or film) obtained with the composition according to the present disclosure is measured by plotting the curve of the tgδ values, obtained during viscoelasticity measurement tests carried out as indicated above, as a function of temperature. The at least one glass transition temperature, Tg, of the polymer corresponds to the temperature at which a local maximum is detected on this curve.

In at least one embodiment, the cosmetic composition according to the present disclosure comprises a dispersion of particles, for example, solid particles, of at least one grafted ethylenic polymer in at least one liquid fatty phase.

The cosmetic composition according to the present disclosure is compatible with keratin materials, for example, with the skin.

As used herein, an “ethylenic” polymer is a polymer obtained by polymerizing monomers comprising an ethylenic unsaturation.

The at least one grafted ethylenic polymer dispersion may be, for example, free of stabilizing polymers other than the grafted ethylenic polymer, such as those described in European Patent No. 749 747, and the particles of grafted ethylenic polymer may therefore not be surface-stabilized by such additional stabilizing polymers. The at least one grafted ethylenic polymer may be therefore dispersed in the at least one liquid fatty phase in the absence of additional stabilizer on the surface of the particles of the at least one grafted ethylenic polymer.

As used herein, a “grafted” polymer is a polymer comprising a skeleton comprising at least one side chain that is pendent or situated at the chain end. In at least one embodiment, the side chain is pendent.

For example, the at least one grafted ethylenic polymer may comprise an ethylenic skeleton which is insoluble in the liquid fatty phase, and side chains bonded covalently to the skeleton which are soluble in the dispersion medium.

The at least one grafted ethylenic polymer may be, for example, a non-crosslinked polymer. For example, the polymer may be obtained by polymerizing monomers comprising a single polymerizable moiety.

In at least one embodiment, the at least one grafted ethylenic polymer may be, for example, a film-forming polymer.

As used herein, a “film-forming” polymer is a polymer suitable for forming, on its own or in the presence of an auxiliary film-forming agent, a continuous film that adheres to a substrate, for example, to keratin materials.

According to one embodiment of the present disclosure, the at least one grafted ethylenic polymer may be, for example, chosen from at least one grafted acrylic polymer.

The at least one grafted ethylenic polymer may be, for example, obtainable by free-radical polymerization in at least one organic polymerization medium:
of at least one ethylenic monomer, for example, at least one acrylic monomer and, optionally, at least one additional, non-acrylic, vinyl monomer, to form the insoluble skeleton; and
of at least one macromonomer comprising a polymerizable end group for forming side chains, wherein the macromonomer has a weight-average molecular mass of greater than or equal to 200, wherein the amount of polymerized macromonomer is present in an amount ranging from 0.05% to 20% by weight of the polymer.

The liquid fatty phase may comprise the organic polymerization medium.

The liquid organic dispersion medium, corresponding to the medium in which the at least one grafted ethylenic polymer is provided may be identical to the polymerization medium.

However, the polymerization medium may be wholly or partly substituted by another liquid organic medium. This other liquid organic medium may be added after polymerization to the polymerization medium. The latter may be, for example, then wholly or partly evaporated.

The liquid fatty phase may comprise organic liquid compounds other than those present in the dispersion medium. These other compounds are chosen such that the at least one grafted ethylenic polymer remains in the state of dispersion in the at least one liquid fatty phase.

The organic liquid dispersion medium may be, for example, present in the at least one liquid fatty phase of the composition according to the disclosure owing to the introduction of the at least one grafted ethylenic polymer dispersion obtained into the composition.

The liquid fatty phase comprises, for example, on a majority basis, at least one liquid organic compound or oil as defined below.

For example, the at least one liquid fatty phase may be a liquid organic phase which may be non-aqueous and water-immiscible at ambient temperature (25° C.).

As used herein, a “liquid organic compound” is a non-aqueous compound which is in the liquid state at ambient temperature (25° C.) and which therefore flows under its own weight.

As used herein, a “silicone compound” is a compound comprising at least one silicon atom.

Among the liquid organic compounds or oils, for example, volatile or non-volatile, that may be present in the liquid organic dispersion medium may be chosen from:
liquid organic compounds, for example, silicone-based or non-silicone-based, having a total solubility parameter according to the Hansen solubility space of less than or equal to 18 (MPa)^{1/2}, for example, less than or equal to 17 (MPa)^{1/2},
monoalcohols having a total solubility parameter according to the Hansen solubility space of less than or equal to 20 (MPa)^{1/2}, and
mixtures thereof.

The total solubility parameter δ according to the Hansen solubility space is defined in the article “Solubility parameter values” by Eric A. Grulke in the work “Polymer Handbook”, 3rd Edition, Chapter VII, p. 519-559, by the relationship: δ=(d_{D}²+d_{P}²+d_{H}²)^{1/2}

wherein:

d_{D} represents the London dispersion forces arising from the formation of dipoles induced during molecular impacts,
d_{P} represents the Debye interaction forces between permanent dipoles, and
d_{H} represents the forces of specific interactions (such as hydrogen bonding, acid/base, donor/acceptor, etc.).

The definition of solvents in the solubility space according to Hansen is described in the article by C. M. Hansen: “The three dimensional solubility parameters”, J. Paint Technol. 39, 105 (1967).

Among the liquid organic compounds, for example, silicone-based or non-silicone-based, having a total solubility parameter according to the Hansen solubility space of less than or equal to 18 (MPa)^{1/2}, non-limiting mention may be made of liquid fatty substances, for example, oils, which may be chosen from natural or synthetic, carbon-based, hydrocarbon-based, fluoro and silicone oils, which are optionally branched, and mixtures thereof.

As used herein, an “oil” is any non-aqueous medium which is liquid at ambient temperature (25° C.) and atmospheric pressure (760 mmHg) and is compatible with application to the skin, mucosae (for example, lips) and/or epidermal derivatives (for example, nails, eyebrows, eyelashes, and hair).

Among these oils, non-limiting mention may be made of plant oils formed from fatty acid esters and from polyols, for example, triglycerides, such as sunflower oil, sesame oil or rapeseed oil, or esters derived from acids or alcohols comprising a long chain (for example, a chain comprising from 6 to 20 carbon atoms), for example, the esters of formula RCOOR′ wherein R is chosen from higher fatty acid residues comprising from 7 to 19 carbon atoms and R′ is chosen from hydrocarbon-based chains comprising from 3 to 20 carbon atoms, such as palmitates, adipates and benzoates, for example, diisopropyl adipate.

Non-limiting mention may also be made of linear, branched and/or cyclic alkanes that may be volatile, for example, liquid paraffin, liquid petroleum jelly or hydrogenated polyisobutylene, isododecane or “Isopars”, volatile isoparaffins. Non-limiting mention may also be made of esters, ethers and ketones.

Non-limiting mention may also be made of silicone oils such as polydimethylsiloxanes and polymethylphenylsiloxanes, optionally substituted with aliphatic and/or aromatic groups, which are optionally fluorinated, or with functional groups such as hydroxyl, thiol and/or amine groups, and volatile silicone oils, which are, for example, cyclic.

For example, non-limiting mention may be made of volatile and/or non-volatile, optionally branched silicone oils.

As used herein, the term “volatile oil” means any non-aqueous medium capable of evaporating from the skin or the lips in less than one hour, and for example, having a vapor pressure, at ambient temperature and atmospheric pressure, ranging from 10⁻³ to 300 mmHg (0.13 Pa to 40 000 Pa).

Volatile silicone oils that may be used in the present disclosure may be chosen from linear and cyclic silicones comprising from 2 to 7 silicon atoms, optionally comprising alkyl or alkoxy groups comprising from 1 to 10 carbon atoms. Non-limiting mention may be made, for example, of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

Non-volatile silicone oils may be chosen, for example, from non-volatile polydimethylsiloxanes (PDMS); polydimethylsiloxanes comprising alkyl, alkoxy or phenyl groups, which are pendent and/or at the end of a silicone chain, these groups comprising from 2 to 24 carbon atoms; phenyl silicones, for instance phenyl trimethicones, phenyl dimethicones, phenyl trimethylsiloxy diphenylsiloxanes, diphenyl dimethicones, diphenyl methyldiphenyltrisiloxanes and polymethylphenylsiloxanes; polysiloxanes modified with fatty acids (for example, C₈-C₂₀), fatty alcohols (for example, C₈-C₂₀) or polyoxyalkylenes (for example, polyoxyethylene and/or polyoxypropylene); amino polysiloxanes; polysiloxanes comprising hydroxyl groups; and fluoro polysiloxanes comprising a fluorinated group that may be pendent or at the end of a silicone chain, comprising from 1 to 12 carbon atoms, all or some of the hydrogens of which are substituted by fluorine atoms; and mixtures thereof.

Non-silicone-based liquid organic compounds with a total solubility parameter according to the Hansen solubility space of less than or equal to 18 (MPa)^{1/2} may be chosen from:
linear, branched and cyclic esters comprising at least 6 carbon atoms, for example, 6 to 30 carbon atoms;
ethers comprising at least 6 carbon atoms, for example, 6 to 30 carbon atoms; and
ketones comprising at least 6 carbon atoms, for example, 6 to 30 carbon atoms.

As used herein, the phrase “liquid monoalcohols having a total solubility parameter according to the Hansen solubility space of less than or equal to 20 (MPa)^{1/2}” means aliphatic fatty liquid monoalcohols comprising from 6 to 30 carbon atoms, the hydrocarbon-based chain not comprising a substitution group. Monoalcohols according to the present disclosure that may be mentioned include, for example, oleyl alcohol, decanol and linoleyl alcohol.

In at least one embodiment, the composition according to the disclosure comprises at least one volatile oil in an amount ranging from 1% to 90% by weight, relative to the total weight of the composition, for example, ranging from 5% to 70% by weight.

In another embodiment, the composition may comprise at least one non-volatile oil in an amount ranging from 0.1% to 80% by weight, relative to the total weight of the composition, for example, ranging from 3% to 50% by weight.

According to at least one embodiment of the present disclosure, the at least one liquid fatty phase may be chosen from at least one non-silicone-based liquid fatty phase.

As used herein, the term “non-silicone-based liquid fatty phase” means a fatty phase comprising at least one non-silicone-based liquid organic compound(s) or oil(s), for example, those mentioned above, wherein the non-silicone compounds are predominantly present in the at least one liquid fatty phase, i.e. to at least 50% by weight, for example, from 50% to 100% by weight, for example, from 60% to 100% by weight (for example from 60% to 99% by weight), or from 65% to 100% by weight (for example from 65% to 95% by weight), relative to the total weight of the at least one liquid fatty phase.

The at least one non-silicone-based liquid organic compound may be, for example, chosen from:
non-silicone-based liquid organic compounds having a total solubility parameter according to the Hansen solubility space of less than or equal to 18 (MPa)^{1/2}, -monoalcohols comprising a total solubility parameter according to the Hansen solubility space of less than or equal to 20 (MPa)^{1/2}; and
mixtures thereof.

The at least one non-silicone-based liquid fatty phase may optionally comprise silicone-based liquid organic compounds or oils, such as those mentioned previously; which may be present in an amount of less than 50% by weight, for example, ranging from 0.1% to 40% by weight, ranging from 1% to 35% by weight, or ranging from 5% to 30% by weight, relative to the total weight of the at least one liquid fatty phase.

In one embodiment of the disclosure, the at least one non-silicone-based liquid fatty phase does not comprise any silicone-based liquid organic compounds or oils.

When the at least one liquid fatty phase is chosen from at least one non-silicone-based liquid fatty phase, the macromonomers present in the at least one grafted ethylenic polymer are for example carbon-based macromonomers as described below.

As used herein, the term “non-silicone-based grafted ethylenic polymer” means a grafted ethylenic polymer predominantly comprising a carbon-based macromonomer and optionally comprising not more than 7% by weight of the total weight of the polymer, for example, not more than 5% by weight, of silicone macromonomer, or even being free of silicone macromonomer.

In another embodiment, the at least one liquid fatty phase may be chosen from at least one silicone-based liquid fatty phase.

The term “silicone-based liquid fatty phase” means a fatty phase comprising at least one silicone-based liquid organic compound or silicone oil such as those described previously, wherein the silicone compounds are predominantly present in the at least one liquid fatty phase, i.e. to at least 50% by weight, for example, from 50% to 100% by weight, for example, from 60% to 100% by weight (for example from 60% to 99% by weight), or even from 65% to 100% by weight (for example from 65% to 95% by weight), relative to the total weight of the at least one liquid fatty phase.

The at least one silicone-based liquid organic compound may be, for example, chosen from:
liquid organic compounds, which are, for example, non-silicone-based or silicone-based, with a total solubility parameter according to the Hansen solubility space of less than or equal to 18 (MPa)^{1/2}.

The at least one silicone-based liquid fatty phase may optionally comprise at least one non-silicone-based liquid organic compound or oil, as described previously, which may be present in an amount of less than 50% by weight, for example, ranging from 0.1% to 40% by weight, for example, from 1% to 35% by weight, and further, for example, ranging from 5% to 30% by weight, relative to the total weight of the at least one liquid fatty phase.

In one embodiment of the disclosure, the at least one silicone-based liquid fatty phase comprises no non-silicone-based liquid organic compounds.

When the at least one liquid fatty phase is a silicone-based liquid fatty phase, the macromonomers present in the at least one grafted ethylenic polymer are, for example, silicone-based macromonomers.

For example, when the at least one liquid fatty phase is a silicone-based liquid fatty phase, the at least one grafted ethylenic polymer present in the composition may be, for example, a silicone-based grafted ethylenic polymer.

The term “silicone-based grafted ethylenic polymer” means a grafted ethylenic polymer predominantly comprising a silicone-based macromonomer optionally comprising up to 7% by weight of the total weight of the polymer, for example, up to 5% by weight, of carbon-based macromonomer, or even being free of carbon-based macromonomer.

The choice of monomers constituting the skeleton of the polymer, of macromonomers, the molecular weight of the polymer, and the proportion of the monomers and macromonomers may be made as a function of the liquid organic dispersion medium so as to obtain, for example, a dispersion of particles of grafted polymer, for example, a stable dispersion.

As used herein, the term “stable dispersion” means a dispersion that is not liable to form a solid deposit or to undergo liquid/solid phase separation, for example, after centrifugation, for example, at 4000 rpm for 15 minutes.

The at least one grafted ethylenic polymer forming the particles in dispersion may comprise a skeleton that is insoluble in the dispersion medium and a portion that is soluble in the dispersion medium.

The at least one grafted ethylenic polymer may be a random polymer.

As used herein, the term “grafted ethylenic polymer” means a polymer obtainable by free-radical polymerization of:
at least one ethylenic monomer,
with at least one macromonomer, in at least one organic polymerization medium.

As used herein, the term “grafted acrylic polymer” means a polymer obtainable by free-radical polymerization of:
at least one acrylic monomer, and optionally of at least one additional non-acrylic vinyl monomer;
with at least one macromonomer, in at least one organic polymerization medium.

The at least one acrylic monomer, for example, may be present in an amount ranging from 50% to 100% by weight, for example from 55% to 100% by weight, further for example from 55% to 95% by weight, still further for example, from 60% to 100% by weight, such as from 60% to 90% by weight, relative to the total weight of the mixture of acrylic monomers and optional non-acrylic vinyl monomers.

For example, the acrylic monomers may be chosen from monomers whose homopolymer is insoluble in the dispersion medium under consideration, i.e. the homopolymer is in solid (or non-dissolved) form at a concentration of greater than or equal to 5% by weight at ambient temperature (20° C.) in the dispersion medium.

As used herein, the term “macromonomer comprising a polymerizable end group” means any polymer comprising on only one of its ends a polymerizable end group capable of reacting during the polymerization reaction with acrylic monomers and optionally the additional non-acrylic vinyl monomers constituting the skeleton. The at least one macromonomer makes it possible to form the side chains of the at least one grafted acrylic polymer. The polymerizable group of the at least one macromonomer may, for example, be an ethylenically unsaturated group capable of free-radical polymerization with the monomers constituting the skeleton.

As used herein, the term “carbon-based macromonomer” means a non-silicone-based macromonomer, for example, an oligomeric macromonomer obtained by polymerization of ethylenically unsaturated non-silicone-based monomer(s), and mainly by polymerization of acrylic and/or non-acrylic vinyl monomers.

As used herein, the term “silicone-based macromonomer” means an organopolysiloxane macromonomer, for example, a polydimethylsiloxane macromonomer.

For example, the at least one macromonomer is chosen from macromonomers whose homopolymer is soluble in the dispersion medium under consideration, i.e. fully dissolved at a concentration of no less than 5% by weight and at ambient temperature in the dispersion medium.

The at least one grafted acrylic polymer may comprise a skeleton (or main chain) comprising a sequence of acrylic units resulting from the polymerization, for example, of at least one acrylic monomer and of side chains (or grafts) derived from the reaction of the macromonomers, wherein the side chains are covalently bonded to the main chain.

The skeleton (or main chain) may be insoluble in the dispersion medium under consideration, whereas the side chains (or grafts) may be soluble in the dispersion medium.

As used herein, the term “acrylic monomers” means monomers chosen from (meth)acrylic acid, (meth)acrylic acid esters (also known as (meth)acrylates), and (meth)acrylic acid amides (also known as (meth)acrylamides).

As acrylic monomers that may be used to form the insoluble skeleton of the polymer, non-limiting mention may be made of the following monomers, used alone or as a mixture, and also the salts thereof:
(i) the (meth)acrylates of formula (I): 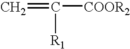
   wherein:
R₁ is chosen from a hydrogen atom and a methyl group;
R₂ is chosen from:
   linear and branched alkyl groups comprising from 1 to 6 carbon atoms, said alkyl groups
      optionally comprising in their chain at least one heteroatom chosen from O, N and S;
      optionally comprising at least one substituent chosen from OH, halogen atoms (F, Cl, Br, or I), and —NR′R″, wherein R′ and R″, which may be identical or different, are chosen from linear and branched C₁-C₄ alkyl groups; and/or
      optionally substituted with at least one polyoxyalkylene group, wherein the polyoxyalkylene group comprises a repetition of from 5 to 30 oxyalkylene units; and
   cyclic alkyl groups comprising from 3 to 6 carbon atoms, said alkyl groups optionally comprising in their chain at least one heteroatom chosen from O, N and S, and/or optionally comprising at least one substituent chosen from OH and halogen atoms (F, Cl, Br, or I).
      Examples of R₂ that may be mentioned include methyl, ethyl, propyl, butyl, isobutyl, methoxyethyl, ethoxyethyl, methoxypolyoxyethylene (350 EO), trifluoroethyl, 2-hydroxyethyl, 2-hydroxypropyl, dimethylaminoethyl, diethylaminoethyl or dimethylaminopropyl groups;
(ii) the (meth)acrylamides of formula: 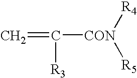
   wherein:
R₃ is chosen from a hydrogen atom and a methyl group;
R₄ and R₅, which may be identical or different, are chosen from:
   hydrogen atoms, and
   linear and branched alkyl groups comprising from 1 to 6 carbon atoms, said alkyl groups optionally comprising at least one substituent chosen from OH, halogen atoms (F, CL, Br, or I), and —NR′R″, wherein R′ and R″, which may be identical or different, are chosen from linear and branched C₁-C₄ alkyl groups; or
R₄ is a hydrogen atom and R₅ is a 1,1-dimethyl-3-oxobutyl group;
   As examples of alkyl groups that may constitute R₄ and R₅, non-limiting mention may be made of n-butyl, t-butyl, n-propyl, dimethylaminoethyl, diethylaminoethyl and dimethylaminopropyl.
(iii) the (meth)acrylic monomers comprising at least one carboxylic, phosphoric or sulphonic acid functional group, for example, acrylic acid, methacrylic acid, acrylamidopropanesulphonic acid.

For example, useful acrylic monomers may be chosen from methyl, ethyl, propyl, butyl and isobutyl (meth)acrylates; methoxyethyl or ethoxyethyl (meth)acrylates; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate; dimethylaminopropylmethacrylamide; (meth)acrylic acid and the salts thereof, and mixtures thereof.

In at least one embodiment, the acrylic monomers may be chosen from methyl acrylate, methoxyethyl acrylate, methyl methacrylate, 2-hydroxyethyl methacrylate, (meth)acrylic acid and dimethylaminoethyl methacrylate, and mixtures thereof.

The at least one additional, non-acrylic, vinyl monomers may be chosen from:
vinyl esters of formula: R₆—COO—CH═CH₂
linear and branched alkyl groups comprising from 1 to 6 carbon atoms,
cyclic alkyl groups comprising from 3 to 6 carbon atoms, and
aromatic groups, for example of benzene, anthracene or naphthalene type;
non-acrylic vinyl monomers comprising at least one functional group chosen from carboxylic, phosphoric and sulphonic acid functional groups, for example, crotonic acid, maleic anhydride, itaconic acid, fumaric acid, maleic acid, styrenesulphonic acid, vinylbenzoic acid, vinylphosphoric acid, and salts thereof; and
non-acrylic vinyl monomers comprising at least one tertiary amine function, such as 2-vinylpyridine or 4-vinylpyridine,
and mixtures thereof.

The at least one acrylic monomer present in the at least one grafted ethylenic polymer, for example, comprise at least one monomer chosen from the (meth)acrylates and the (meth)acrylamides described above in sections (i) and (ii). The acrylic monomers may, for example, comprise at least one monomer chosen from C₁-C₃ alkyl (meth)acrylates.

The at least one grafted ethylenic polymer may comprise (meth)acrylic acid.

According to one embodiment of the disclosure, the at least one grafted ethylenic polymer does not comprise any acid-functional monomer, for example, does not comprise (meth)acrylic acid.

For example, among the salts, those that may be mentioned are those obtained by neutralization of acidic moieties with inorganic bases such as sodium hydroxide, potassium hydroxide or ammonium hydroxide, or organic bases such as alkanolamines, for instance monoethanolamine, diethanolamine, triethanolamine or 2-methyl-2-amino-1-propanol.

Non-limiting mention may also be made of the salts formed by neutralization of tertiary amine units, for example using a mineral or organic acid. Among the mineral acids that may be mentioned are sulfuric acid, hydrochloric acid, hydrobromic acid, hydriodic acid, phosphoric acid and boric acid. Among the organic acids that may be mentioned are acids comprising at least one group chosen from carboxylic, sulfonic and phosphonic groups. They may be linear, branched or cyclic aliphatic acids, or aromatic acids. These acids may also comprise at least one heteroatom chosen from O and N, for example in the form of hydroxyl groups. Acetic acid or propionic acid, terephthalic acid, and citric acid and tartaric acid may be mentioned.

According to one embodiment of the disclosure, the at least one grafted ethylenic polymer comprises no additional non-acrylic vinyl monomers. In this embodiment, the insoluble skeleton of the at least one grafted ethylenic polymer is formed solely from acrylic monomers.

The non-polymerized acrylic monomers may be soluble in the dispersion medium under consideration, but the polymer formed with these monomers is insoluble in the dispersion medium.

According to one embodiment of the disclosure, the at least one grafted ethylenic polymer is obtainable by free-radical polymerization in at least one organic polymerization medium of:
at least one principal acrylic monomer chosen from at least one C₁-C₃ alkyl (meth)acrylate, and optionally of at least one additional acrylic monomer chosen from acrylic acid, methacrylic acid and the alkyl (meth)acrylates of formula (I) defined above, and salts thereof, to form the insoluble skeleton; and
of at least one silicone-based macromonomer comprising a polymerizable end group, as defined above.

As a principal acrylic monomer it is possible to use methyl acrylate, methyl methacrylate, ethyl acrylate, ethyl methacrylate, n-propyl acrylate, n-propyl methacrylate, isopropyl acrylate and isopropyl methacrylate, and mixtures thereof.

The principal acrylic monomer, may be, for example, chosen from methyl acrylate, methyl methacrylate and ethyl methacrylate.

The additional acrylic monomers may be chosen from:
(meth)acrylic acid and its salts, and
the (meth)acrylates of formula (I) and their salts: 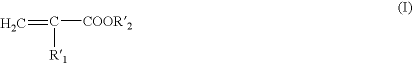
   wherein:
   R′₁ is chosen from a hydrogen atom and a methyl group;
   R′₂ is chosen from
      linear and branched alkyl groups comprising from 1 to 6 carbon atoms and comprising in their chain at least one oxygen atom, and/or optionally comprising at least one substituent chosen from —OH, halogen atoms (F, Cl, Br, I) and —NR′R″, wherein R′ and R″, which may be identical or different, may be chosen from linear and branched C₁-C₃ alkyls; and
      a cyclic alkyl group comprising from 3 to 6 carbon atoms, optionally comprising at least one oxygen atom in the cyclic alkyl group and/or optionally comprising at least one substituent chosen from —OH and halogen atoms (F, Cl, Br, I),
and mixtures thereof.

R′₂ may be chosen from, for example, methoxyethyl, ethoxyethyl, trifluoroethyl; 2-hydroxyethyl, 2-hydroxypropyl, dimethylaminoethyl, diethylaminoethyl, and dimethylaminopropyl.

Among the additional acrylic monomers non-limiting mention may be made, for example, of (meth)acrylic acid, methoxyethyl or ethoxyethyl (meth)acrylates; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate, salts thereof, and mixtures thereof.

The at least one macromonomer comprises at one of the ends of the chain a polymerizable end group capable of reacting during the polymerization with the acrylic monomers and optionally the additional vinyl monomers, to form the side chains of the at least one grafted ethylenic polymer. The polymerizable end group may, for example, be a vinyl or (meth)acrylate (or (meth)acryloyl) group, for example, a (meth)acrylate group.

The at least one macromonomer may be chosen from macromonomers whose homopolymer has a glass transition temperature (Tg) of no more than 25° C., for example, ranging from −100° C. to 25° C. for example, ranging from −80° C. to 0° C.

The at least one macromonomer may have a weight-average molecular mass of greater than or equal to 200, for example, greater than or equal to 300, further, for example, greater than or equal to 500 and even further, for example, greater than 600.

For example, the macromonomers have a weight-average molecular mass (Mw) ranging from 200 to 100 000, for example, ranging from 500 to 50 000, ranging from 800 to 20 000, ranging from 800 to 10 000 and ranging from 800 to 6000.

As used herein, the weight-average (Mw) and number-average (Mn) molar masses are determined by liquid gel permeation chromatography (THF solvent, calibration curve established with linear polystyrene standards, refractometric detector).

Carbon-based macromonomers that may, for example, be mentioned include:
(i) homopolymers and copolymers chosen from linear and branched C₈-C₂₂ alkyl (meth)acrylates, comprising a polymerizable end group chosen from vinyl and (meth)acrylate groups, among which non-limiting mention may be made of: poly(2-ethylhexyl acrylate) macromonomers with a mono(meth)acrylate end group; poly(dodecyl acrylate) or poly(dodecyl methacrylate) macromonomers with a mono(meth)acrylate end group; poly(stearyl acrylate) or poly(stearyl methacrylate) macromonomers with a mono(meth)acrylate end group.

Such macromonomers are described, for example, in European Patent Nos. EP 895 467 and EP 96 459, and in the article by Gillman K. F., Polymer Letters, Vol 5, pages 477-481 (1967).

Non-limiting mention may be made, for example, of macromonomers based on poly(2-ethylhexyl acrylate) or poly(dodecyl acrylate) with a mono(meth)acrylate end group;
(ii) polyolefins comprising an ethylenically unsaturated end group, for example, comprising a (meth)acrylate end group. Examples of such polyolefins that may be mentioned include the following macromonomers comprising a (meth)acrylate end group: polyethylene macromonomers, polypropylene macromonomers, macromonomers of polyethylene/polypropylene copolymer, macromonomers of polyethylene/polybutylene copolymer, polyisobutylene macromonomers; polybutadiene macromonomers; polyisoprene macromonbmers; polybutadiene macromonomers; poly(ethylene/butylene)polyisoprene macromonomers.

Such macromonomers are described, for example, in U.S. Pat. No. 5,625,005, which mentions ethylene/butylene and ethylene/propylene macromonomers comprising a (meth)acrylate reactive end group.

Non-limiting mention may be made, for example, of the poly(ethylene/butylene) methacrylate such as that sold under the name Kraton Liquid L-1253 by Kraton Polymers.

Silicone-based macromonomers that may be mentioned, for example, include polydimethylsiloxanes with a mono(meth)acrylate end group, for example, those of formula (II): 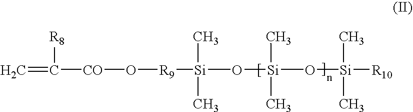
R₈ is chosen from a hydrogen atom and a methyl group;
R₉ is chosen from divalent hydrocarbon-based groups comprising from 1 to 10 carbon atoms, optionally comprising one or two ether bonds —O—;
R₁₀ is chosen from alkyl groups comprising from 1 to 10 carbon atoms, for example, from 2 to 8 carbon atoms;
n is chosen from a number ranging from 1 to 300, for example, ranging from 3 to 200 and further, for example, ranging from 5 to 100.

For example, silicone-based macromonomers that may be used include monomethacryloxypropyl polydimethylsiloxanes such as those sold under the name PS560-K6 by the company United Chemical Technologies Inc. (UCT) or under the name MCR-M17 by the company Gelest Inc.

For example, the polymerized macromonomer (constituting the side chains of the at least one grafted ethylenic polymer) may be present in an amount ranging from 0.1% to 15% by weight of the total weight of the polymer, for example, from 0.2% to 10% by weight and further, for example, from 0.3% to 8% by weight.

For example, the at least one grafted ethylenic polymer dispersed in a non-silicone-based liquid fatty phase, may be obtained by polymerization:
of methyl acrylate/acrylic acid monomers and of a polyethylene/polybutylene macromonomer with a methacrylate end group (for example, Kraton L-1253), for example, in a solvent chosen from isododecane, isononyl isononanoate, octyldodecanol, diisostearyl malate, an alkyl benzoate (such as Finsolv TN).

For example, the at least one grafted ethylenic polymer dispersed in a silicone-based liquid fatty phase, may be obtained by polymerization:
of methyl acrylate and the monomethacryloyloxypropyl polydimethylsiloxane macromonomer with a weight-average molecular weight ranging from 800 to 6000, for example, in decamethylcyclopentasiloxane.

The weight-average molecular mass (Mw) of the at least one grafted ethylenic polymer may range from 10 000 to 300 000, such as from 20 000 to 200 000 and further such as from 25 000 to 150 000.

In a given organic dispersion medium, the polymers of the disclosure have the capacity of folding over on themselves, thus forming particles of substantially spherical shape, the periphery of these particles comprising the deployed side chains, which ensure the stability of these particles. Such particles resulting from the characteristics of the at least one grafted ethylenic polymer may have, for example, the feature of not agglomerating in the medium and thus self-stabilized and of forming a stable polymer particle dispersion.

For example, the at least one grafted acrylic polymers of the dispersion are capable of forming nanometer-sized particles, with a mean size ranging from 10 to 400 nm for example, from 20 to 200 nm.

As a result of this very small size, the at least one grafted ethylenic polymer particles in dispersion may be stable and therefore have little susceptibility to form agglomerates.

The dispersion of grafted ethylenic polymer may thus be a dispersion that is stable and does not form sediments when it is placed at ambient temperature (25° C.) for an extended period (for example 24 hours).

For example, the dispersion of grafted ethylenic polymer particles has a solids content (or dry extract) of polymer of from 40% to 70% by weight of solids, for example, from 45% to 65% by weight.

The dispersion of grafted ethylenic polymer particles may be prepared by a process comprising a free-radical copolymerization step, in an organic polymerization medium, of at least one acrylic monomer with at least one macromonomer.

In one embodiment, the liquid organic dispersion medium may be identical to or different from the polymerization medium.

For example, the copolymerization may be performed conventionally in the presence of a polymerization initiator. The polymerization initiators may be free-radical initiators. In general, such a polymerization initiator may be chosen from organic peroxide compounds such as dilauroyl peroxide, dibenzoyl peroxide or tert-butyl peroxy-2-ethylhexanoate; diazo compounds such as azobisisobutyronitrile or azobisdimethylvaleronitrile.

The reaction may also be initiated using photoinitiators or with radiation such as UV or neutrons, or with plasma.

In general, to perform this process, at least a portion of the organic polymerization medium, a portion of the additional acrylic and/or vinyl monomers, which will constitute the insoluble skeleton after polymerization, all of the macromonomer (which will constitute the side chains of the polymer) and a portion of the polymerization initiator are introduced into a reactor whose size is suitable for the amount of polymer to be prepared. At this stage of introduction, the reaction medium forms a relatively homogeneous medium.

The reaction medium is then stirred and heated up to a temperature to obtain polymerization of the monomers and macromonomers. After a certain time, the initially homogeneous and clear medium leads to a dispersion of milky appearance. A mixture consisting of the remaining portion of monomers and of polymerization initiator is then added. After an adequate time during which the mixture is heated with stirring, the medium stabilizes in the form of a milky dispersion, the dispersion comprising polymer particles stabilized in the medium wherein they have been created, the stabilization being due to the presence, in the polymer, of side chains that are soluble in the dispersion medium.

The at least one grafted ethylenic polymer present in the composition according to the disclosure, for example, exhibits at least one glass transition temperature ranging from 0° C. to 80° C., for example, from 0° C. to 60° C., and further, for example, from 0° C. to 40° C. The glass transition temperature is measured, for example, in accordance with the protocol described above relating to the deposit obtained with the composition, employing, for example, solely the dispersion of grafted ethylenic polymer; the glass transition temperature is therefore measured on a film which comprises only the at least one grafted ethylenic polymer.

The at least one grafted ethylenic polymer described above may be present in the composition according to the disclosure in an amount ranging from 0.5% to 45% by weight, relative to the total weight of the composition, for example, ranging from 1% to 30% by weight and further, for example, ranging from 2% to 25% by weight.

The at least one grafted ethylenic polymer may be, for example, present in the non-volatile fraction of the composition in an amount greater than or equal to 20% by weight of the non-volatile fraction. By “non-volatile fraction” of the composition is meant the entirety of the constituents present in the composition that are not volatile. A volatile compound is a compound which, taken in isolation, has a non-zero vapor pressure which, at ambient temperature (25° C.) and atmospheric pressure, ranges, for example, from 10⁻³ to 300 mmHg (0.133 Pa to 40 000 Pa). For example, the amount of grafted ethylenic polymer in the non-volatile fraction of the composition may range from 20% to 90% by weight of the non-volatile fraction, for example, ranging from 20% to 70% by weight, and further, for example, from 20% to 60% by weight, and even further, for example, from 30% to 60% by weight.

For example, the non-volatile fraction of the composition corresponds in fact to the mixture of constituents which remains on the skin after the full drying of the composition which has been applied to the skin.

The composition according to the disclosure may comprise at least one colorant chosen from water-soluble dyes and pulverulent colorants such as pigments, nacres and flakes. The colorants may be present in the composition in an amount ranging from 0.01% to 50% by weight, relative to the weight of the composition, for example, from 0.01% to 30% by weight.

As used herein, “pigments” means white or colored, mineral or organic particles of any form which are insoluble in the physiological medium and are intended for coloring the composition.

As used herein, “nacres” refers to iridescent particles of any form, produced, for example, by certain mollusks within their shell, or else synthesized.

The pigments may be white or colored, mineral and/or organic. Among mineral pigments, non-limiting mention may be made of titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders such as aluminum powder and copper powder.

Among organic pigments non-limiting mention may be made of carbon black, D & C pigments, and lakes based on cochineal carmine, barium, strontium, calcium and aluminum.

Non-limiting mention may also be made of effect pigments, such as particles comprising an organic or mineral, natural or synthetic substrate, for example glass, acrylic resins, polyester, polyurethane, polyethylene terephthalate, ceramics or aluminas, the substrate being bare or covered with metallic substances such as aluminum, gold, silver, platinum, copper, bronze, or with metal oxides such as titanium dioxide, iron oxide, chromium oxide and mixtures thereof.

The nacreous pigments may be chosen from white nacreous pigments such as titanium-coated mica, or bismuth oxychloride, colored nacreous pigments such as titanium mica coated with iron oxides, titanium mica coated with, for example, ferric blue or chromium oxide, titanium mica coated with an organic pigment of the aforementioned type, and also nacreous pigments based on bismuth oxychloride. Nacreous pigments may also be chosen from interference pigments, for example, liquid-crystal pigments or multilayer pigments.

For example, the at least one grafted ethylenic polymer present in the composition according to the disclosure makes it possible to obtain a good homogeneous dispersion of the pulverulent colorants, such as the pigments and nacres.

Another embodiment of the present disclosure is a foundation composition comprising a dispersion of particles of at least one grafted ethylenic polymer in at least one liquid fatty phase as described above, and at least one colorant, for example, pigments and nacres, or any other filler having an optical effect.

The water-soluble dyes are, for example, beet root juice and methylene blue.

The composition according to the disclosure may further comprise at least one filler, for example, in an amount ranging from 0.01% to 50% by weight, relative to the total weight of the composition, for example, ranging from 0.01% to 30% by weight. As used herein, “filler” refers to particles of any shape, colorless or white, mineral or synthetic, which are insoluble in the medium of the composition irrespective of the temperature at which the composition is manufactured. The at least one filler serves, for example, to modify the rheology or texture of the composition.

The at least one filler may be mineral or organic fillers of any form, platelet-like, spherical or oblong, irrespective of the crystallographic form (for example, leaflet, cubic, hexagonal, orthorhombic, etc.). Non-limiting mention may be made of talc, mica, silica, kaolin, polyamide (Nylon®) powders (Orgasol® from Atochem), poly-β-alanine powders and polyethylene powders, powders of polymers of tetrafluoroethylene (Teflon®), lauroyllysine, starch, boron nitride, hollow polymeric microspheres such as those of polyvinylidene chloride/acrylonitrile, for instance Expancel® (Nobel Industries), and of acrylic acid copolymers (Polytrap® from Dow Corning), and silicone resin microbeads (Tospearls® from Toshiba, for example), particles of elastomeric organopolysiloxanes, precipitated calcium carbonate, magnesium carbonate and bicarbonate, hydroxyapatite, hollow silica microspheres (Silica Beads® from Maprecos), glass or ceramic microcapsules, metal soaps derived from organic carboxylic acids comprising 8 to 22 carbon atoms, for example, 12 to 18 carbon atoms, for example zinc stearate, magnesium stearate or lithium stearate, zinc laurate and magnesium myristate.

The composition according to the disclosure may further comprise ingredients commonly used in cosmetology, such as vitamins, moisturizers, emollients, free-radical scavengers, thickeners, trace elements, softeners, sequestering agents, perfumes, alkalifying or acidifying agents, preservatives, sunscreens, surfactants, antioxidants, gums, waxes, propellants, or mixtures thereof.

The person skilled in the art will of course take care to select this or these optional additional compounds, and/or their amount, wherein the advantageous properties of the corresponding composition according to the disclosure are not, or not substantially, adversely affected by the intended addition.

The composition according to the disclosure may be in a from chosen from suspensions, dispersions, solutions, gels, emulsions, for example, an oil-in-water (O/W) or water-in-oil (W/O) emulsion, or multiple (W/O/W or polyol/O/W or O/W/O) emulsion; creams, pastes, mousses, dispersions of vesicles, for example, of ionic or nonionic lipids, two-phase or multiphase lotions, sprays, powders and pastes. The composition may be anhydrous: for example, it may comprise a stick or an anhydrous paste. The composition may be a non-rinse composition.

A person skilled in the art may select the appropriate galenical form, and also the method of preparing it, on the basis of his or her general knowledge, taking into account firstly the nature of the constituents used, for example, their solubility in the vehicle, and secondly the intended application of the composition.

According to another embodiment, the disclosure also relates to a cosmetic apparatus comprising:
i) a container comprising at least one compartment, wherein the container is closed by a closing member; and
ii) a composition within the at least one compartment comprising a dispersion of particles of at least one grafted ethylenic polymer in at least one liquid fatty phase, wherein the at least one grafted ethylenic polymer is present in an amount sufficient to allow the composition to form a deposit having a complex storage modulus E* of less than or equal to 200 MPa.

The container may be in any appropriate form. It may, for example, be in the form of a bottle, a tube, a jar, a case, a box, a sachet or a carton.

The closing member may be in the form of a removable stopper, a lid, a cap, a tear-off strip or a capsule, for example, of the type comprising a body attached to the container and a cover cap articulated on the body. It may also be in the form of a member for selectively closing the container, for example, a pump, a valve or a flap valve.

The container may be combined with an applicator, for example, in the form of a block of foam or of elastomer, a felt or a spatula. The applicator may be free (tuft or sponge) or securely fastened to a rod borne by the closing member, as described, for example, in U.S. Pat. No. 5,492,426. The applicator may be securely fastened to the container, as described, for example, in French Patent No. FR 2 761 959.

The product may be contained directly in the container, or indirectly. By way of example, the product may be arranged on an impregnated support, for example, in the form of a wipe or a pad, and arranged (individually or in plurality) in a box or in a sachet. Such a support incorporating the product is described, for example, in PCT Patent Application No. WO 01/03538.

The closing member may be coupled to the container by screwing. In one embodiment, the coupling between the closing member and the container is done other than by screwing, for example, via a bayonet mechanism, by snap fastening, gripping, welding, adhesive bonding or by magnetic attraction. As used herein, the term “snap fastening” means any system involving the crossing of a bead or cord of material by elastic deformation of a portion, for example, of the closing member, followed by return to the elastically unconstrained position of the portion after the crossing of the bead or cord.

The container may be at least partially made of at least one thermoplastic material. Examples of thermoplastic materials that may be mentioned include polypropylene or polyethylene.

In one embodiment, the container is made of at least one non-thermoplastic material, for example, glass or metal (or alloy).

The container may have rigid walls or deformable walls, for example, in the form of a tube or a tubular bottle.

The container may comprise means for distributing or facilitating the distribution of the composition. By way of example, the container may have deformable walls so as to allow the composition to exit in response to a positive pressure inside the container, this positive pressure is caused by elastic (or non-elastic) squeezing of the walls of the container. In one embodiment, for example, when the product is in the form of a stick, the product may be driven out by a piston mechanism. Still in the case of a stick, for example, of makeup product (lipstick, foundation, etc.), the container may comprise a mechanism, for example, a rack mechanism, a threaded-rod mechanism or a helical groove mechanism, and may be capable of moving a stick in the direction of the aperture. Such a mechanism is described, for example, in French Patent No. FR 2 806 273 or in French Patent No. FR 2 775 566. Such a mechanism for a liquid product is described in French Patent No. FR 2 727 609.

The container may be comprised of a carton with a base comprising at least one housing comprising the composition, and a lid, for example, articulated on the base, and capable of at least partially covering the base. Such a carton is described, for example, in PCT Patent Application No. WO 03/018423 or in French Patent No. FR 2 791 042.

The container may be equipped with a drainer arranged in the region of the aperture of the container. Such a drainer makes it possible to wipe the applicator and possibly the rod to which it may be securely fastened. Such a drainer is described, for example, in French Patent No. FR 2 792 618.

The composition may be at atmospheric pressure inside the container (at room temperature) or pressurized, for example, by means of a propellant gas (aerosol). In the latter case, the container is equipped with a valve (of the type used for aerosols).

The content of the patents or patent applications mentioned above is incorporated by reference into the present disclosure.

The present invention will now be described in more detail in the light of the following examples, which are given by way of illustration and not of limitation.

These examples illustrate the preparation of polymers in accordance with the present disclosure that are suitable for forming a dispersion of particles in an organic medium under consideration. In these examples, following preparation of the dispersion, the weight-average (Mw) and number-average (Mn) molar masses of the polymer, the glass transition temperature of the polymer, the solids content (or dry extract) of the dispersion and the size of the polymer particles were determined.

The weight-average (Mw) and number-average (Mn) molar masses were determined by liquid gel-permeation chromatography (THF solvent, calibration curve established with linear polystyrene standards, refractometric detector).

The measurement of the glass transition temperature (Tg) was performed according to standard ASTM D3418-97, by differential thermal analysis (DSC “Differential Scanning Calorimetry”) on a calorimeter, over a temperature range from −100° C. to +150° C., at a heating rate of 10° C./minute in 150 μl aluminum crucibles.

The crucibles were prepared in the following manner: 100 μl of the dispersion obtained were introduced into a 150 μl aluminum crucible and the solvent was allowed to evaporate over 24 hours at ambient temperature and at 50% relative humidity. The operation was repeated and the crucible was then introduced into a Mettler DSC30 calorimeter.

The solids content (or dry extract), i.e. the amount of non-volatile matter, may be measured in various ways: non-limiting mention may be made, for example, of the methods by oven-drying or the methods by drying by exposure to infrared radiation.

The solids content was measured by heating the sample with infrared rays with a wavelength of from 2 μm to 3.5 μm. The substances contained in the composition that had a high vapor pressure evaporated under the effect of this radiation. Measuring the weight loss of the sample made it possible to determine the dry extract of the composition. These measurements were performed using an LP16 commercial infrared desiccator from Mettler. This technique is fully described in the documentation for the instrument supplied by Mettler.

The measuring protocol was as follows: about 1 g of the composition was spread onto a metal cup. After the cup was introduced into the desiccator, it was subjected to a nominal temperature of 120° C. for an hour. The wet mass of the sample, corresponding to the initial mass, and the dry mass of the sample, corresponding to the mass after exposure to the radiation, were measured using a precision balance.

The solids content was calculated in the following manner:
dry extract=100×(dry mass/wet mass).

The particle sizes may be measured by various techniques: non-limiting mention may be made, for example, of light-scattering techniques (dynamic and static), Coulter counter methods, sedimentation rate measurements (related to the size via Stokes' law) and microscopy. These techniques make it possible to measure a particle diameter and, for some of them, a particle size distribution.

The sizes and size distributions of the particles in the compositions according to the disclosure were measured by static light scattering using a commercial granulometer such as the MasterSizer 2000 from Malvern. The data were processed on the basis of the Mie scattering theory. This theory, which is exact for isotropic particles, makes it possible to determine an “effective” particle diameter in the case of non-spherical particles. This theory is described, for example, in the work by Van de Hulst, H. C., “Light Scattering by Small Particles”, Chapters 9 and 10, Wiley, New York, 1957.

The composition was characterized by its mean “effective” diameter by volume D[4.3], defined in the following manner:

$$D [ 4.3 ] = \frac{{\sum }_{i} {V}_{i} \cdot {d}_{i}}{{\sum }_{i} {V}_{i}}$$

wherein Vᵢ is chosen from the volume of the particles with an effective diameter dᵢ. This parameter is described, for example, in the technical documentation of the granulometer.

The measurements were performed at 25° C. on a dilute particle dispersion, obtained from the composition in the following manner: 1) dilution by a factor of 100 with water, 2) homogenization of the solution, 3) standing of the solution for 18 hours, 4) recovery of the whitish uniform supernatant.

As used herein, the “effective” diameter was obtained by taking a refractive index of 1.33 for water and a mean refractive index of 1.42 for the particles.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contain certain errors necessarily resulting from the standard deviation found in their respective testing measurements.

The embodiments disclosed herein are illustrated in greater detail by the examples described below.

### EXAMPLE 1

A dispersion of particles of a polymer in isododecane was prepared, the polymer was obtained by polymerization of methyl acrylate and the at least one macromonomer corresponding to a polyethylene/polybutylene copolymer with a methacrylate end group (Kraton L-1253).

A reactor was charged with 2 kg of heptane, 2 kg of isododecane, 2.8 kg of methyl acrylate and 1.2 kg of macromonomer of the polyethylene/polybutylene copolymer type with a methacrylate end group (Kraton L-1253) and 320 g of tert-butyl peroxy-2-ethylhexanoate (Trigonox 21S).

The reaction mixture was stirred and heated at ambient temperature to 90° C. over 1 hour. After 15 minutes at 90° C., a change was observed in the appearance of the reaction medium, which passed from a transparent appearance to a milky appearance. Heating with stirring was continued for a further 15 minutes, and then a mixture consisting of 16 kg of methyl acrylate and 200 g of Trigonox 21S was added dropwise over 1 hour.

Thereafter the mixture was heated at 90° C. for 4 hours and then the heptane was distilled from the reaction medium. The outcome of this distillation operation was a stable dispersion of polymer particles thus prepared in isododecane.

The at least one grafted ethylenic polymer comprised 6% by weight of macromonomer relative to the weight of the polymer.

The characteristics of the polymer and of the particles formed by the polymer were as follows:
weight-average molecular mass Mw=119 900
number-average molecular mass Mn=16 300
polydispersity index (Mw/Mn)=7.37
glass transition: 10° C. by Mettler DSC;
dry extract: 52.4% in isododecane, carried out by thermal balance;
particle size: 46 nm with polydispersity of 0.05, performed on a Malvern Autosizer Lo-C at 25° C.

### EXAMPLE 2

This example illustrates the preparation of a polymer forming a dispersion of particles in a silicone oil, the polymer was obtained by polymerization of methyl acrylate and of the monomethacryloyloxypropyl polydimethylsiloxane macromonomer comprising a weight-average molecular weight of 5000, sold under the name MCR-M17 by Gelest Inc.

200 g of heptane, 200 g of decamethylcyclopentasiloxane, 30 g of methyl acrylate, 10 g of monomethacryloyloxypropyl polydimethylsiloxane MCR-M17 and 3.2 g of tert-butyl peroxy-2-ethylhexanoate (Trigonox 21S) were loaded into a 1 liter reactor.

The reaction mixture was stirred and heated to 90° C. over 1 hour. After 15 minutes at 90° C., a change was observed in the appearance of the reaction medium, which passed from a transparent appearance to a milky appearance. Heating with stirring was continued for a further 15 minutes, and a mixture consisting of 160 g of methyl acrylate and 2 g of Trigonox 21S was then added dropwise over 1 hour. Next, the mixture was heated for 4 hours at 90° C. and the heptane was then distilled from the reaction medium.

The outcome of this distillation operation was a stable dispersion of particles of polymer thus prepared in decamethylcyclopentasiloxane (D5).

The at least one grafted ethylenic polymer comprised 5% by weight of macromonomer (and hence of side chains soluble in D5) relative to the weight of the polymer.

The characteristics of the polymer and of the particles formed by the polymer were as follows:
weight-average molecular mass Mw=102 347
number-average molecular mass Mn=28 283
polydispersity index (Mw/Mn)=3.62
dry extract: 51.4% in D5, carried out by thermal balance
glass transition: 12° C. by Mettler DSC
particle size: 160 nm with polydispersity of 0.04, performed on a Malvern Autosizer Lo-C at 25° C.

Following implementation of the stability protocol in accordance with Example 1, the dispersion was found to be stable.

The polymers of Examples 1 and 2 comprised the following monomers, their proportions are indicated as a percentage by weight of the polymer:

| Polymer | Ex. 1 | Ex. 2 |
|---|---|---|
| Methyl acrylate | 94 | 95 |
| Carbon-based | 6 | — |
| macromonomer | | |
| Silicone-based | — | 5 |
| macromonomer | | |

### EXAMPLE 3

A foundation was prepared with the following composition (the amounts are indicated as a percentage relative to the total weight of the composition):

| | Example |
|---|---|
| | 3 |
| Abil EM 97 | 0.9 |
| Inwitor 780 K | 0.3 |
| Isododecane | 37.4 |
| Grafted ethylenic | Ex. 1 |
| polymer dispersion | 26.8 |
| Kraton G 1701E | 2.8 |
| D5 | 5 |
| Pigments | 12 |
| Nylon powder | 1.1 |
| Polyethylene wax | 1.4 |
| Perfume | 0.3 |
| Water | 7 |
| Preservatives | 1 |
| Octane-1,2-diol | 0.3 |
| Glycerol | 3 |
| Magnesium sulphate | 0.7 |

The composition comprised 15.2% by weight of grafted ethylenic polymer active substance.

The amount of grafted ethylenic polymer in the non-volatile fraction of the composition was 39% by weight.

Ingredients Used:

ABIL EM 97: ethoxylated propoxylated α,ω-substituted silicone/cyclomethicone mixture (85/15) sold by the Goldschmidt company
Inwitor 780 K: mono- and diglycerides of isostearic acid, esterified with succinic acid, sold by the Sasol company
Kraton G1701 E: styrene/ethylene-propylene diblock copolymer, sold by the company Kraton Polymer
D5: cyclopentadimethylsiloxane
Nylon powder sold under the name Orgasol® 2002 extra D NAT COS by the company Atofina
Polyethylene wax sold under the name ceridust 9205F by the company Clariant

The complex modulus E*, the damping power tgδ and the glass transition temperature of the deposit obtained with the composition were measured in accordance with the measuring protocol described above. The results obtained were as follows:

| | Example |
|---|---|
| | 3 |
| Complex storage | 34 |
| modulus E* (MPa) | |
| Damping power tgδ | 1.3 |
| Tg (° C.) | 26 |

Moreover, this foundation according to the disclosure, when applied to the skin, allowed makeup to be obtained which exhibited good properties of comfort, of non-stickiness and of non-transfer.

### EXAMPLE 4

A foundation was prepared which was similar to that of Example 3, the at least one grafted ethylenic polymer dispersion of Example 1 is replaced by that of Example 2.

This gave a composition which forms a film as disclosed herein. The foundation, when applied to the skin, lead to skin makeup which was comfortable, non-sticky and exhibits good non-transfer properties.

1. A cosmetic skincare or makeup composition comprising a dispersion of particles of at least one grafted ethylenic polymer in at least one liquid fatty phase, wherein the at least one grafted ethylenic polymer is present in an amount sufficient to allow the composition to form a deposit comprising a complex storage modulus E* of less than or equal to 200 MPa.

2. A composition according to claim 1, wherein the complex storage modulus E* is less than or equal to 50 MPa.

3. A composition according to claim 1, wherein the at least one grafted ethylenic polymer is present in an amount sufficient to allow the composition to form a deposit comprising a damping power of greater than or equal to 0.7 tgδ.

4. A composition according to claim 3, wherein the damping power is greater than or equal to 1.1 tgδ.

5. A composition according to claim 1, wherein the at least one grafted ethylenic polymer is present in an amount sufficient to allow the composition to form a deposit comprising at least one glass transition temperature ranging from 0 to 40° C.

6. A composition according to claim 1, wherein the composition comprises a non-volatile fraction and wherein the at least one grafted ethylenic polymer is present in an amount of greater than or equal to 20% by weight, relative to the weight of the non-volatile fraction.

7. A composition according to claim 6, wherein the at least one grafted ethylenic polymer is present in an amount ranging from 20% to 90% by weight, relative to the total weight of the non-volatile fraction of the composition.

8. A composition according to claim 7, wherein the at least one grafted ethylenic polymer is present in an amount ranging from 20% to 60% by weight, relative to the total weight of the non-volatile fraction of the composition.

9. A composition according to claim 1, wherein the at least one grafted ethylenic polymer comprises
an ethylenic skeleton which is insoluble in the at least one liquid fatty phase, and
side chains bonded covalently to the skeleton and soluble in the at least one liquid fatty phase.

10. A composition according to claim 1, wherein the at least one grafted ethylenic polymer is chosen from grafted acrylic polymers.

11. A composition according to claim 1, wherein the at least one grafted ethylenic polymer is dispersed in the absence of additional stabilizer on the surface of the particles of the at least one grafted ethylenic polymer.

12. A composition according to claim 1, wherein the at least one grafted ethylenic polymer is chosen from acrylic polymers obtainable by free-radical polymerization in at least one organic polymerization medium:
of at least one acrylic monomer, and, optionally, at least one additional, non-acrylic, vinyl monomer, to form the insoluble skeleton; and
of at least one macromonomer comprising a polymerizable end group for forming side chains, wherein the at least one macromonomer has a weight-average molecular mass of no less than 200, and wherein the at least one polymerized macromonomer is present in an amount ranging from 0.05% to 20% by weight of the polymer.

13. A composition according to claim 12, wherein the at least one acrylic monomer is chosen from the following monomers, and their salts:
(i) (meth)acrylates of formula: 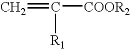
wherein:
R₁ is chosen from a hydrogen atom and a methyl group;
R₂ is chosen from:
linear and branched alkyl groups comprising from 1 to 6 carbon atoms, said alkyl groups
optionally comprising in their chain at least one heteroatom chosen from O, N and S;
optionally comprising at least one substituent chosen from OH, halogen atoms, and —NR′R″, wherein R′ and R″, which may be identical or different, are chosen from linear and branched C₁-C₄ alkyl groups; and/or
optionally substituted with at least one polyoxyalkylene group, wherein the polyoxyalkylene group comprises a repetition of from 5 to 30 oxyalkylene units; and
cyclic alkyl groups comprising from 3 to 6 carbon atoms, said alkyl groups optionally comprising in their chain at least one heteroatom chosen from O, N and S, and/or optionally comprising at least one substituent chosen from OH and halogen atoms,
(ii) (meth)acrylamides of formula: 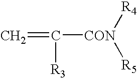
wherein:
R₃ is chosen from a hydrogen atom and a methyl group;
R₄ and R₅, which may be identical or different, are chosen from:
hydrogen atoms, and
linear and branched alkyl groups comprising from 1 to 6 carbon atoms, said alkyl groups optionally comprising at least one substituent chosen from OH, halogen atoms, and —NR′R″, wherein R′ and R″, which may be identical or different, are chosen from linear and branched C₁-C₄ alkyl groups; or
R₄ is a hydrogen atom and R₅ is a 1,1-dimethyl-3-oxobutyl group;
(iii) (meth)acrylic monomers comprising at least one functional group chosen from carboxylic, phosphoric and sulphonic acid functional groups.

14. A composition according to claim 12, wherein the at least one acrylic monomer is chosen from methyl (meth)acrylates, ethyl (meth)acrylates, propyl (meth)acrylates, butyl (meth)acrylates and isobutyl (meth)acrylates; methoxyethyl (meth)acrylates; ethoxyethyl (meth)acrylates; trifluoroethyl methacrylate; dimethylaminoethyl methacrylate, diethylaminoethyl methacrylate, 2-hydroxypropyl (meth)acrylate, 2-hydroxyethyl (meth)acrylate; dimethylaminopropylmethacrylamide; (meth)acrylic acid, and the salts thereof.

15. A composition according to claim 12, wherein the at least one acrylic monomer is chosen from methyl acrylate, methoxyethyl acrylate, methyl methacrylate, 2-hydroxyethyl methacrylate, (meth)acrylic acid and dimethylaminoethyl methacrylate.

16. A composition according to claim 12, wherein the at least one grafted ethylenic polymer comprises (meth)acrylic acid.

17. A composition according to claim 13, wherein the at least one acrylic monomer comprises at least one monomer chosen from (meth)acrylates of (i) and (meth)acrylamides of (ii).

18. A composition according to claim 17, wherein the at least one acrylic monomer comprises at least one monomer chosen from C₁-C₃ alkyl (meth)acrylates.

19. A composition according to claim 12, wherein the at least one grafted acrylic polymer does not comprise acid-functional monomers.

20. A composition according to claim 12, wherein the at least one grafted acrylic polymer does not comprise additional, non-acrylic, vinyl monomers.

21. A composition according to claim 12, wherein the at least one grafted acrylic polymer is obtainable by free radical polymerization of at least one acrylic monomer and of at least one additional non-acrylic, vinyl monomer, and of the at least one macromonomer.

22. A composition according to claim 12, wherein the at least one additional, non-acrylic, vinyl monomer is chosen from:
vinyl esters of formula: R₆—COO—CH═CH₂
wherein R₆ is chosen from
linear and branched alkyl groups comprising from 1 to 6 carbon atoms,
cyclic alkyl groups comprising from 3 to 6 carbon atoms, and
aromatic groups;
non-acrylic vinyl monomers comprising at least one functional group chosen from carboxylic, phosphoric and sulphonic acid functional groups, and
non-acrylic vinyl monomers comprising at least one tertiary amine function.

23. A composition according to claim 12, wherein the at least one acrylic monomer is present in an amount ranging from 50% to 100% by weight, relative to the weight of the mixture of acrylic monomers and optional non-acrylic vinyl monomers.

24. A composition according to claim 23, wherein the at least one acrylic monomer is present in an amount ranging from 70% to 100% by weight, relative to the weight of the mixture of acrylic monomers and optional non-acrylic vinyl monomers.

25. A composition according to claim 12, wherein the at least one macromonomer comprises at one of the ends of the chain a polymerizable end group chosen from vinyl groups and (meth)acrylate groups.

26. A composition according to claim 12, wherein the at least one macromonomer has a weight-average molecular mass of greater than or equal to 300.

27. A composition according to claim 26, wherein the at least one macromonomer has a weight-average molecular mass of greater than 600.

28. A composition according to claim 12, wherein the at least one macromonomer has a weight-average molecular mass (Mw) ranging from 200 to 100 000.

29. A composition according to claim 28, wherein the at least one macromonomer has a weight-average molecular mass (Mw) ranging from 800 to 6000.

30. A composition according to claim 12, wherein the polymerized macromonomer is present in an amount ranging from 0.1% to 15% by weight, relative to the total weight of the polymer.

31. A composition according to claim 30, wherein the polymerized macromonomer is present in an amount ranging from 0.3% to 8% by weight, relative to the total weight of the polymer.

32. A composition according to claim 1, wherein the at least one liquid fatty phase comprises at least one liquid organic compound chosen from:
liquid organic compounds comprising a total solubility parameter according to the Hansen solubility space of less than or equal to 18 (MPa)^{1/2}; and
monoalcohols comprising a total solubility parameter according to the Hansen solubility space of less than equal to 20 (MPa)^{1/2}.

33. A composition according to claim 32, wherein the at least one liquid organic compound is chosen from at least one non-volatile oil.

34. A composition according to claim 1, wherein the at least one liquid fatty phase is chosen from at least one non-silicone-based liquid fatty phase.

35. A composition according to claim 34, wherein the at least one non-silicone-based liquid fatty phase comprises at least 50% by weight of at least one non-silicone-based organic liquid compound chosen from:
non-silicone-based organic liquid compounds comprising a total solubility parameter according to the Hansen solubility space of less than or equal to 18 (MPa)^{1/2}; and
liquid monoalcohols comprising a total solubility parameter according to the Hansen solubility space of less than or equal to 20 (MPa)^{1/2}.

36. A composition according to claim 34, wherein the at least one non-silicone-based liquid fatty phase comprises less than 50% by weight of at least one silicone-based liquid organic compound comprising a total solubility parameter according to the Hansen solubility space of less than or equal to 18 (MPa)^{1/2}.

37. A composition according to claim 34, wherein the non-silicone-based liquid fatty phase comprises no silicone-based liquid organic compounds.

38. A composition according to claim 12, wherein the at least one macromonomer is chosen from carbon-based macromonomers.

39. A composition according to claim 38, wherein the carbon-based macromonomers are chosen from:
(i) homopolymers and copolymers of linear and branched C₈-C₂₂ alkyl acrylates and methacrylates, comprising a polymerizable end group chosen from vinyl groups and (meth)acrylate groups; and
(ii) polyolefins comprising a polymerizable ethylenically unsaturated end group.

40. A composition according to claim 38, wherein the carbon-based macromonomers are chosen from:
(i) poly(2-ethylhexyl acrylate) macromonomers with a mono(meth)acrylate end group; poly(dodecyl acrylate) macromonomers with a mono(meth)acrylate end group; poly(dodecyl methacrylate) macromonomers; poly(stearyl acrylate) macromonomers with a mono(meth)acrylate end group; and poly(stearyl methacrylate) macromonomers with a mono(meth)acrylate end group;
(ii) polyethylene macromonomers, polypropylene macromonomers, macromonomers of polyethylene/polypropylene copolymer, macromonomers of polyethylene/polybutylene copolymer, polyisobutylene macromonomers, polybutadiene macromonomers, polyisoprene macromonomers, polybutadiene macromonomers, poly(ethylene/butylene)polyisoprene macromonomers, wherein the end group is chosen from a (meth)acrylate end group.

41. A composition according to claim 38, wherein the carbon-based macromonomers are chosen from:
(i) poly(2-ethylhexyl acrylate) macromonomers with a mono(meth)acrylate end group, and poly(dodecyl acrylate) macromonomers with a mono(meth)acrylate end group; and
(ii) poly(ethylene/butylene) methacrylate.

42. A composition according to claim 12, wherein the at least one grafted ethylenic polymer is obtained by polymerization of methyl acrylate and of a polyethylene/polybutylene macromonomer with a methacrylate end group.

43. A composition according to claim 42, wherein the polymerization occurs in a solvent chosen from isododecane, isononyl isononanoate, octyidodecanol, diisostearyl malate, and a C₁₂-C₁₅ alkyl benzoate.

44. A composition according to claim 34, wherein the at least one grafted ethylenic polymer is chosen from non-silicone-based grafted ethylenic polymers.

45. A composition according to claim 44, wherein the at least one non-silicone-based grafted ethylenic polymer comprises predominantly a carbon-based macromonomer which optionally comprises not more than 7% by weight of silicone-based macromonomer, relative to the total weight of the polymer.

46. A composition according to claim 44, wherein the non-silicone-based grafted ethylenic polymer is free from carbon-based macromonomer.

47. A composition according to claim 1, wherein the at least one liquid fatty phase is a silicone-based liquid fatty phase.

48. A composition according to claim 47, wherein the silicone-based liquid fatty phase comprises at least 50% by weight of at least one silicone-based organic liquid compound chosen from at least one silicone-based organic liquid compounds comprising a total solubility parameter according to the Hansen solubility space of less than or equal to 18 (MPa)^{1/2}.

49. A composition according to claim 36, wherein the at least one silicone-based organic liquid compound comprises at least one volatile silicone oil.

50. A composition according to claim 48, wherein the at least one silicone-based organic liquid compound comprises at least one volatile silicone oil.

51. A composition according to claim 49, wherein the at least one volatile silicone oil is chosen from octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, and decamethyltetrasiloxane.

52. A composition according claim 50, wherein the at least one volatile silicone oil is chosen from octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, and decamethyltetrasiloxane.

53. A composition according to claim 36, wherein the at least one silicone-based organic liquid compound comprises at least one non-volatile silicone oil.

54. A composition according to claim 48, wherein the silicone-based organic liquid compound comprises at least one non-volatile silicone oil.

55. A composition according to claim 53, wherein the at least one non-volatile silicone oil is chosen from non-volatile polydialkylsiloxanes; polydimethylsiloxanes comprising alkyl, alkoxy or phenyl groups which comprise from 2 to 24 carbon atoms and which are pendent and/or at the end of a silicone chain; phenyl silicones; polysiloxanes modified with groups chosen from fatty acids, fatty alcohols and polyoxyalkylenes; amino polysiloxanes; polysiloxanes comprising hydroxyl groups; and fluoro polysiloxanes comprising a fluorinated group that is pendent and/or at the end of a silicone chain, comprising from 1 to 12 carbon atoms, and wherein at least one hydrogen of which is substituted by at least one fluorine atom.

56. A composition according to claim 54, wherein the at least one non-volatile silicone oil is chosen from non-volatile polydialkylsiloxanes; polydimethylsiloxanes comprising alkyl, alkoxy or phenyl groups which comprise from 2 to 24 carbon atoms and which are pendent and/or at the end of a silicone chain; phenyl silicones; polysiloxanes modified with groups chosen from fatty acids, fatty alcohols and polyoxyalkylenes; amino polysiloxanes; polysiloxanes comprising hydroxyl groups; and fluoro polysiloxanes comprising a fluorinated group that is pendent and/or at the end of a silicone chain, comprising from 1 to 12 carbon atoms, and wherein at least one hydrogen of which is substituted by at least one fluorine atom.

57. A composition according to claim 47, wherein the at least one liquid fatty phase comprises less than 50% by weight of at least one non-silicone-based liquid organic compound.

58. A composition according to claim 35, wherein the at least one non-silicone-based liquid organic compound is chosen from non-silicone-based liquid organic compounds comprising a total solubility parameter according to the Hansen solubility space of less than 18 (MPa)^{1/2}; and liquid monoalcohols comprising a total solubility parameter according to the Hansen solubility space of less than or equal to 20 (MPa)^{1/2.}

59. A composition according to claim 58, wherein the at least one non-silicone-based organic liquid compound comprising a total solubility parameter according to the Hansen solubility space of less than 18 (MPa)^{1/2} is chosen from carbon-based, hydrocarbon-based and fluoro oils; linear, branched and/or cyclic alkanes, optionally volatile; esters; ketones; and ethers.

60. A composition according to claim 57, wherein the at least one non-silicone-based liquid organic compound is chosen from non-silicone-based liquid organic compounds comprising a total solubility parameter according to the Hansen solubility space of less than 18 (MPa)^{1/2}; and liquid monoalcohols comprising a total solubility parameter according to the Hansen solubility space of less than or equal to 20 (MPa)^{1/2}.

61. A composition according to claim 60, wherein the at least one non-silicone-based organic liquid compound comprising a total solubility parameter according to the Hansen solubility space of less than 18 (MPa)^{1/2} is chosen from carbon-based, hydrocarbon-based and fluoro oils, linear, branched and/or cyclic alkanes, optionally volatile; esters; ketones; and ethers.

62. A composition according to claim 32, wherein the monoalcohols comprising a total solubility parameter according to the Hansen solubility space of less than or equal to 20 (MPa)^{1/2} are chosen from aliphatic fatty monoalcohols comprising from 6 to 30 carbon atoms, wherein the hydrocarbon chain comprises no substitution group.

63. A composition according to claim 58, wherein the monoalcohols comprising a total solubility parameter according to the Hansen solubility space of less than or equal to 20 (MPa)^{1/2} are chosen from aliphatic fatty monoalcohols comprising from 6 to 30 carbon atoms, wherein the hydrocarbon chain comprises no substitution group.

64. A composition according to claim 1 wherein the at least one liquid fatty phase comprises at least one non-silicone-based volatile oil.

65. A composition according to claim 64, wherein the at least one non-silicone-based volatile oil is chosen from isododecane, isodecane and isohexadecane.

66. A composition according to claim 47, wherein the at least one liquid fatty phase comprises no non-silicone-based liquid organic compounds.

67. A composition according to claim 33, wherein the at least one non-volatile oil is present in an amount ranging from 0.1% to 80% by weight, relative to the total weight of the composition.

68. A composition according to claim 67, wherein the at least one non-volatile oil is present in an amount ranging from 3% to 50% by weight, relative to the total weight of the composition.

69. A composition according to claim 1, comprising at least one volatile oil in an amount ranging from 1% to 90% by weight, relative to the total weight of the composition.

70. A composition according to claim 69, comprising at least one volatile oil in an amount ranging from 5% to 70% by weight, relative to the total weight of the composition.

71. A composition according to claim 12, wherein the at least one macromonomer is chosen from silicone-based macromonomers.

72. A composition according to claim 71, wherein the silicone-based macromonomers are chosen from organopolysiloxane macromonomers.

73. A composition according to claim 72, wherein the organopolysiloxane macromonomer is a polydimethylsiloxane monomer.

74. A composition according to claim 73, wherein the at least one macromonomer is chosen from polydimethylsiloxanes with a mono(meth)acrylate end group.

75. A composition according to claim 71, wherein the at least one silicone-based macromonomer is chosen from macromonomers of formula (II): 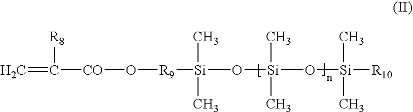
wherein
R₈ is chosen from a hydrogen atom and a methyl group;
R₉ is chosen from divalent hydrocarbon-based groups comprising from 1 to 10 carbon atoms and optionally comprises one or two ether bonds —O—;
R₁₀ is chosen from alkyl groups comprising from 1 to 10 carbon atoms; and
n is an integer ranging from 1 to 300.

76. A composition according to claim 75, wherein R₁₀ is chosen from alkyl groups comprising from 2 to 8 carbon atoms.

77. A composition according to claim 75, wherein n ranges from 5 to 100.

78. A composition according to claim 12, wherein the at least one grafted acrylic polymer is obtainable by free-radical polymerization in the at least one polymerization medium:
of at least one principal acrylic monomer chosen from at least one C₁-C₃ alkyl (meth)acrylates, and optionally of at least one additional acrylic monomer chosen from acrylic acid, methacrylic acid and alkyl (meth)acrylates of formula (I): 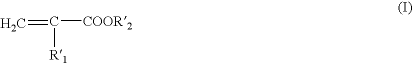
wherein:
R′₁ is chosen from a hydrogen atom and a methyl group;
R′₂ is chosen from
linear and branched alkyl groups comprising from 1 to 6 carbon atoms, wherein the alkyl groups comprise in their chain at least one oxygen atom, and/or optionally comprise at least one substituent chosen from —OH, halogen atoms and —NR′R″, where R′ and R″, which may be identical or different, are chosen from linear and branched C₁-C₃ alkyl groups; and
cyclic alkyl groups comprising from 3 to 6 carbon atoms, optionally comprising in their chain at least one oxygen atom in the cyclic alkyl group and/or optionally comprising at least one substituent chosen from —OH and halogen atoms;
and salts thereof, to form the insoluble skeleton; and
of at least one silicone-based macromonomer.

79. A composition according to claim 78, wherein R′₂ is chosen from methoxyethyl, ethoxyethyl, trifluoroethyl; 2-hydroxyethyl, 2-hydroxypropyl, dimethylaminoethyl, diethylaminoethyl, and dimethylaminopropyl.

80. A composition according to claim 78, wherein the at least one principal acrylic monomer is chosen from methyl (meth)acrylate, ethyl (meth)acrylate, n-propyl (meth)acrylate, and isopropyl (meth)acrylate.

81. A composition according to claim 78, wherein the at least one principal acrylic monomer is chosen from methyl acrylate, methyl methacrylate and ethyl methacrylate.

82. A composition according to claim 78, wherein the at least one additional acrylic monomer is chosen from (meth)acrylic acid, methoxyethyl (meth)acrylate, ethoxyethyl (meth)acrylate, trifluoroethyl methacrylate, dimethylaminoethyl methacrylate, diethylaminoethyl methacrylate, 2-hydroxypropyl (meth)acrylate, 2-hydroxyethyl (meth)acrylate, and salts thereof.

83. A composition according to claim 78, wherein the at least one additional acrylic monomer is chosen from acrylic acid and methacrylic acid.

84. A composition according to claim 12, wherein the at least one grafted ethylenic polymer is chosen from the polymers obtained by polymerizing:
methyl acrylate and monomethacryloyloxypropyl polydimethylsiloxane macromonomer comprising a weight-average molecular weight ranging from 800 to 6000, optionally in a polymerization medium chosen from decamethylcyclopentasiloxane or phenyltrimethicone.

85. A composition according to claim 12, wherein the at least one grafted ethylenic polymer is chosen from at least one silicone-based grafted ethylenic polymer.

86. A composition according to claim 85, wherein the at least one silicone-based grafted ethylenic polymer comprises predominantly a silicone-based macromonomer, optionally comprising not more than 7% by weight of carbon-based macromonomer, based on the total weight of the polymer.

87. A composition according to claim 85, wherein the at least one silicone-based grafted ethylenic polymer is free from carbon-based macromonomer.

88. A composition according to claim 1, wherein the at least one grafted ethylenic polymer has a weight-average molecular mass (Mw) ranging from 10 000 to 300 000.

89. A composition according to claim 88, wherein the at least one grafted ethylenic polymer has a weight-average molecular mass (Mw) ranging from 25 000 to 150 000.

90. A composition according to claim 1, wherein the particles of grafted ethylenic polymer have an average size ranging from 10 to 400 nm.

91. A composition according to claim 90, wherein the particles of grafted ethylenic polymer have an average size ranging from 20 to 200 nm.

92. A composition according to claim 1, wherein the at least one grafted ethylenic polymer is chosen from at least one film-forming polymer.

93. A composition according to claim 1, wherein the at least one grafted ethylenic polymer has at least one glass transition temperature ranging from 0° C. to 80° C.

94. A composition according to claim 93, wherein the at least one grafted ethylenic polymer has at least one glass transition temperature ranging from 0° C. to 40° C.

95. A composition according to claim 1, wherein the at least one grafted ethylenic polymer is present in an amount ranging from 0.5% to 45% by weight, relative to the total weight of the composition.

96. A composition according to claim 95, wherein the at least one grafted ethylenic polymer is present in an amount ranging from 2% to 25% by weight, relative to the total weight of the composition.

97. A composition according to claim 1, further comprising at least one colorant.

98. A composition according to claim 97, wherein the at least one colorant is a pulverulent colorant.

99. A composition according to claim 98, wherein the pulverulent colorant is chosen from pigments and nacres.

100. A composition according to claim 1, further comprising at least one cosmetic ingredient chosen from vitamins, moisturizers, emollients, free-radical scavengers, thickeners, trace elements, softeners, sequesterants, perfumes, alkalifying or acidifying agents, preservatives, sunscreens, surfactants, antioxidants, gums, waxes, and propellants.

101. A cosmetic composition according to claim 1, wherein the composition is in a form chosen from suspensions, dispersions, solutions, gels, emulsions, creams, pastes, mousses, vesicle dispersions, two-phase or multiphase lotions, sprays and powders.

102. A cosmetic composition according to claim 101, wherein the emulsions are chosen from oil-in-water (O/W), water-in-oil (W/O), and multiple (W/O/W or polyol/O/W or O/W/O) emulsions.

103. A cosmetic composition according to claim 1, wherein said composition is in anhydrous form.

104. A composition according to claim 1, wherein the composition is a skin makeup product.

105. A foundation comprising at least one composition comprising a dispersion of particles of at least one grafted ethylenic polymer in at least one liquid fatty phase, wherein the at least one grafted ethylenic polymer is present in an amount sufficient to allow the composition to form a deposit comprising a complex storage modulus E* of less than or equal to 200 MPa.

106. A foundation composition comprising
a dispersion of particles of at least one grafted ethylenic polymer in at least one liquid fatty phase, wherein the at least one grafted ethylenic polymer is present in an amount sufficient to allow the composition to form a deposit comprising a complex storage modulus E* of less than or equal to 200 MPa, and
at least one colorant.

107. A cosmetic apparatus comprising:
a) a container comprising at least one compartment, wherein the container is closed by a closing member; and
b) a composition within the at least one compartment comprising a dispersion of particles of at least one grafted ethylenic polymer in at least one liquid fatty phase, wherein the at least one grafted ethylenic polymer is present in an amount sufficient to allow the composition to form a deposit comprising a complex storage modulus E* of less than or equal to 200 MPa.

108. A cosmetic apparatus according to claim 107, wherein the at least one container is formed at least partly of at least one thermoplastic material.

109. A cosmetic apparatus according to claim 107, wherein the at least one container is formed at least partly of at one non-thermoplastic material.

110. A cosmetic apparatus according to claim 107, wherein, with the container in its closed position, the closing member is screwed onto the container.

111. An apparatus according to claim 107, wherein, with the container in its closed position, the closing member is coupled with the container other than by screwing.

112. An apparatus according to claim 107, wherein the composition is substantially at atmospheric pressure within the compartment.

113. An apparatus according to claim 107, wherein the composition is pressurized within the container.

114. A process of making up and/or caring for the skin comprising applying to the skin a cosmetic composition comprising a dispersion of particles of at least one grafted ethylenic polymer in at least one liquid fatty phase, wherein the at least one grafted ethylenic polymer is present in an amount sufficient to allow the composition to form a deposit comprising a complex storage modulus E* of less than or equal to 200 MPa.

115. A process for obtaining a deposit on skin comprising applying to the skin a composition comprising a dispersion of particles of at least one grafted ethylenic polymer in at least one liquid fatty phase, wherein the at least one grafted ethylenic polymer is present in an amount sufficient to allow the composition to form a deposit comprising a complex storage modulus E* of less than or equal to 200 MPa, and wherein the deposit has at least one property chosen from good adhesion, comfort and good transfer resistance.
